(19) United States
(12) Patent Application Publication
(10) Pub. No.: US 2006/0064083 A1
(43) Pub. Date: Mar. 23, 2006

(54) Multi-tip probe used for an ocular procedure

(76) Inventors: Steve Khalaj, Laguna Hills, CA (US); Dorin Panescu, San Jose, CA (US)

Correspondence Address: IRELL & MANELLA LLP, 840 NEWPORT CENTER DRIVE, SUITE 400, NEWPORT BEACH, CA, 92660, US

(21) Appl. No.: 10/943,615

(22) Filed: Sep. 17, 2004

(51) Int. Cl. A61B 18/14

(52) U.S. Cl. 606/41; 606/50

(57) ABSTRACT

An apparatus and method for denaturing corneal tissue. The apparatus includes a first electrode and a second electrode that are both inserted into a cornea. The electrodes are coupled to a power unit that delivers energy sufficient to denature corneal tissue. The dual electrode assembly allows for the creation of multiple denatured spots with a single application of energy. Additionally, the multi-electrode assembly provides uniform spacing between the denatured spots.

### BACKGROUND OF THE INVENTION

1. Field of the Invention

The present invention relates to a method and apparatus for treating ocular tissue.

2. Prior Art

Techniques for correcting vision have included reshaping the cornea of the eye. For example, myopic conditions can be corrected by cutting a number of small incisions in the corneal membrane. The incisions allow the corneal membrane to relax and increase the radius of the cornea. The incisions are typically created with either a laser or a precision knife. The procedure for creating incisions to correct myopic defects is commonly referred to as radial keratotomy and is well known in the art.

Radial keratotomy techniques generally make incisions that penetrate approximately 95% of the cornea. Penetrating the cornea to such a depth increases the risk of puncturing the Descemets membrane and the endothelium layer, and creating permanent damage to the eye. Additionally, light entering the cornea at the incision sight is refracted by the incision scar and produces a glaring effect in the visual field. The glare effect of the scar produces impaired night vision for the patient.

The techniques of radial keratotomy are only effective in correcting myopia. Radial keratotomy cannot be used to correct an eye condition such as hyperopia. Additionally, keratotomy has limited use in reducing or correcting an astigmatism. The cornea of a patient with hyperopia is relatively flat (large spherical radius). A flat cornea creates a lens system which does not correctly focus the viewed image onto the retina of the eye. Hyperopia can be corrected by reshaping the eye to decrease the spherical radius of the cornea. It has been found that hyperopia can be corrected by heating and denaturing local regions of the cornea. The denatured tissue contracts and changes the shape of the cornea and corrects the optical characteristics of the eye. The procedure of heating the corneal membrane to correct a patient's vision is commonly referred to as thermokeratoplasty.

U.S. Pat. No. 4,461,294 issued to Baron; U.S. Pat. No. 4,976,709 issued to Sand and PCT Publication WO 90/12618, all disclose thermokeratoplasty techniques which utilize a laser to heat the cornea. The energy of the laser generates localized heat within the corneal stroma through photonic absorption. The heated areas of the stroma then shrink to change the shape of the eye.

Although effective in reshaping the eye, the laser based systems of the Baron, Sand and PCT references are relatively expensive to produce, have a non-uniform thermal conduction profile, are not self limiting, are susceptible to providing too much heat to the eye, may induce astigmatism and produce excessive adjacent tissue damage, and require long term stabilization of the eye. Expensive laser systems increase the cost of the procedure and are economically impractical to gain widespread market acceptance and use.

Additionally, laser thermokeratoplasty techniques non-uniformly shrink the stroma without shrinking the Bowmans layer. Shrinking the stroma without a corresponding shrinkage of the Bowmans layer, creates a mechanical strain in the cornea. The mechanical strain may produce an undesirable reshaping of the cornea and probable regression of the visual acuity correction as the corneal lesion heals. Laser techniques may also perforate Bowmans layer and leave a leucoma within the visual field of the eye.

U.S. Pat. Nos. 4,326,529 and 4,381,007 issued to Doss et al, disclose electrodes that are used to heat large areas of the cornea to correct for myopia. The electrode is located within a sleeve that suspends the electrode tip from the surface of the eye. An isotropic saline solution is irrigated through the electrode and aspirated through a channel formed between the outer surface of the electrode and the inner surface of the sleeve. The saline solution provides an electrically conductive medium between the electrode and the corneal membrane. The current from the electrode heats the outer layers of the cornea. Heating the outer eye tissue causes the cornea to shrink into a new radial shape. The saline solution also functions as a coolant which cools the outer epithelium layer.

The saline solution of the Doss device spreads the current of the electrode over a relatively large area of the cornea. Consequently, thermokeratoplasty techniques using the Doss device are limited to reshaped corneas with relatively large and undesirable denatured areas within the visual axis of the eye. The electrode device of the Doss system is also relatively complex and cumbersome to use.

“A Technique for the Selective Heating of Corneal Stroma” Doss et al., Contact & Intraoccular Lens Medical Jrl., Vol. 6, No. 1, pp. 13-17, January-March, 1980, discusses a procedure wherein the circulating saline electrode (CSE) of the Doss patent was used to heat a pig cornea. The electrode provided 30 volts r.m.s. for 4 seconds. The results showed that the stroma was heated to 70° C. and the Bowman's membrane was heated 45° C., a temperature below the 50-55° C. required to shrink the cornea without regression.

“The Need For Prompt Prospective Investigation” McDonnell, Refractive & Corneal Surgery, Vol. 5, January/February, 1989 discusses the merits of corneal reshaping by thermokeratoplasty techniques. The article discusses a procedure wherein a stromal collagen was heated by radio frequency waves to correct for a keratoconus condition. As the article reports, the patient had an initial profound flattening of the eye followed by significant regression within weeks of the procedure.

“Regression of Effect Following Radial Thermokeratoplasty in Humans” Feldman et al., Refractive and Corneal Surgery, Vol. 5, September/October, 1989, discusses another thermokeratoplasty technique for correcting hyperopia. Feldman inserted a probe into four different locations of the cornea. The probe was heated to 600° C. and was inserted into the cornea for 0.3 seconds. Like the procedure discussed in the McDonnell article, the Feldman technique initially reduced hyperopia, but the patients had a significant regression within 9 months of the procedure.

Refractec, Inc. of Irvine Calif., the assignee of the present application, has developed a system to correct hyperopia with a thermokeratoplasty probe that is connected to a console. The probe includes a tip that is inserted into the stroma layer of a cornea. Electrical current provided by the console flows through the eye to denature the collagen tissue within the stroma. The process of inserting the probe tip and applying electrical current can be repeated in a circular pattern about the cornea. The denatured tissue will change the refractive characteristics of the eye. The procedure is taught by Refractec under the service marks CONDUCTIVE KERATOPLASTY and CK.

A CK procedure typically requires a number of single applications with a uni-polar tip. By way of example, a procedure may require 24 separate denatured spots on the cornea. Sequentially inserting the tip and delivering energy can be a relatively time consuming process. Additionally, it is desirable to have relatively uniform spacing between denatured spots along the same radian. It would be desirable to provide an electrode assembly that can reduce the time required to create the denatured spots in a CK procedure and provide uniform spacing between spots.

### BRIEF SUMMARY OF THE INVENTION

A method and apparatus for denaturing corneal tissue. The apparatus includes a first electrode and a second electrode that are inserted into a cornea. Energy is delivered by one or both electrodes to denature corneal tissue.

### BRIEF DESCRIPTION OF THE DRAWINGS

FIG. 1 is a perspective view of a system for denaturing corneal tissue;

FIG. 2 is an enlarged view of a bi-polar electrode assembly of the system;

FIG. 3 is a graph showing a waveform that is provided by a console of the system;

FIG. 4 is an enlarged view of a pair of electrode tips inserted into a cornea;

FIG. 5 is top view showing a pattern of denatured spots in a cornea;

FIG. 6 is an alternate embodiment of an electrode assembly with three electrodes;

FIG. 7 is an alternate embodiment of an electrode assembly having three separate stops;

FIG. 8 is an alternate embodiment of an electrode assembly having pairs of electrode tips;

FIG. 9 is an alternate embodiment of an electrode assembly having a radial pattern of electrode tips;

FIG. 10 is an alternate embodiment of a system with a lid speculum ground element.

### DETAILED DESCRIPTION

Disclosed is an apparatus and method for denaturing corneal tissue. The apparatus includes a first electrode and a second electrode that are both inserted into a cornea. The electrodes are coupled to a power unit that delivers energy sufficient to denature corneal tissue. The dual electrode assembly allows for the creation of multiple denatured spots with a single application of energy. Additionally, the multi-electrode assembly provides uniform spacing between the denatured spots.

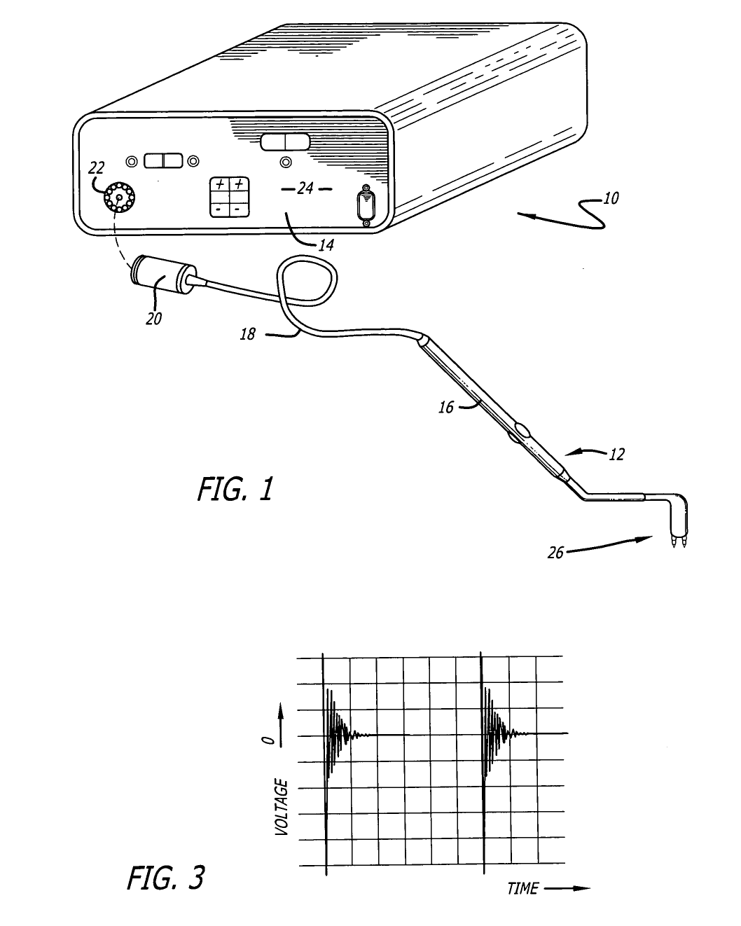

Referring to the drawings more particularly by reference numbers, FIG. 1 shows an embodiment of an apparatus 10 that can be used to denature corneal tissue. The apparatus 10 includes an electrode probe 12 coupled to a console 14. The console 14 contains a power supply that can deliver electrical power to the probe 12. The probe 12 has a hand piece 16 and wires 18 that couple the probe electrode to a connector 20 that plugs into a mating receptacle 22 located on the front panel 24 of the console 14. The hand piece 16 may be constructed from a non-conductive material. The probe 12 includes a multi-electrode assembly 26.

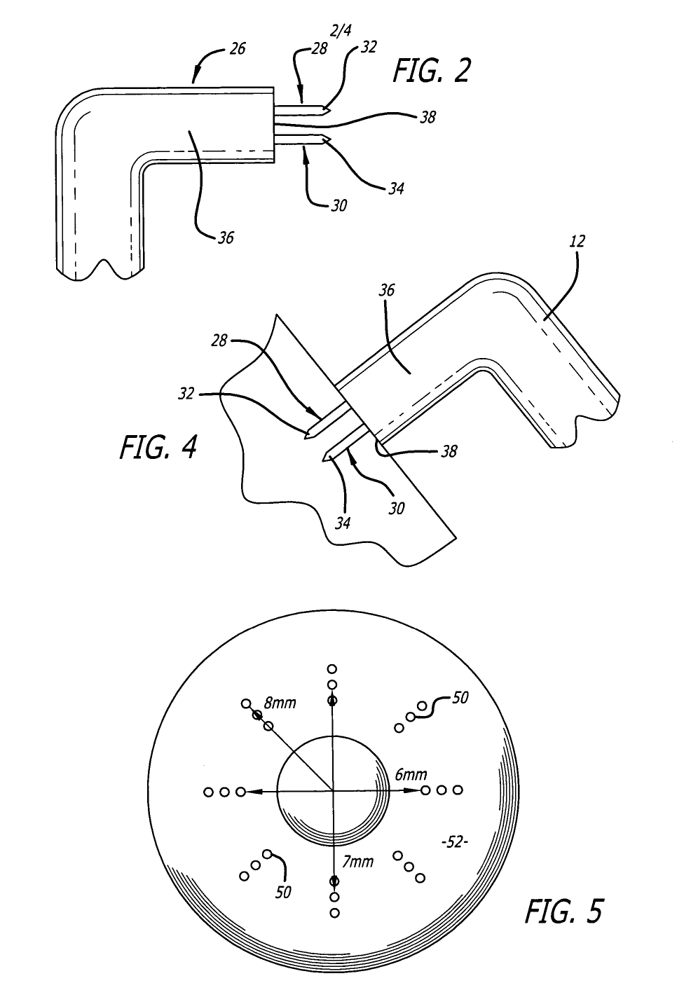

As shown in FIG. 2, the multi-electrode assembly 26 may include a first electrode 28 and a second electrode 30. By way of example, the electrodes 28 and 30 may be separated 0.2 to 2.0 millimeters center to center. The electrodes 28 and 30 can be generally described as being co-planar, as opposed to co-axial. The electrodes 28 and 30 may include pointed tips 32 and 34, respectively, that extend from a housing 36. The tips 32 and 34 are typically constructed from a metal material. The housing 36 is typically constructed from a dielectric material such as plastic. For example, the dielectric material may be a polyofelin polymer. Alternatively, the housing 36 may be constructed to include a hollow metal filled with a dielectric material. The housing 36 may have a bottom surface 38 that functions as a stop to limit the penetration depth of the tips 32 and 34 into a cornea. Alternatively, the bottom surface 38 may be formed by a separate part or a separate member of housing 36. As an example, a Teflon stop can be coupled to the housing 36 to form bottom surface 36.

The console 14 may provide a predetermined amount of energy, through a controlled application of power for a predetermined time duration. The console 14 may have manual controls that allow the user to select treatment parameters such as the power and time duration. The console 14 can also be constructed to provide an automated operation. The console 14 may have monitors and feedback systems for measuring physiologic tissue parameters such as tissue impedance, tissue temperature and other parameters, and adjust the output power of the radio frequency amplifier to accomplish the desired results.

In one embodiment, the console 14 provides voltage limiting to prevent arcing. To protect the patient from overvoltage or overpower, the console 14 may have an upper voltage limit and/or upper power limit which terminates power to the probe when the output voltage or power of the unit exceeds a predetermined value.

The console 14 may also contain monitor and alarm circuits which monitors physiologic tissue parameters such as the resistance or impedance of the load and provides adjustments and/or an alarm when the resistance/impedance value exceeds and/or falls below predefined limits. The adjustment feature may change the voltage, current, and/or power delivered by the console such that the physiological parameter is maintained within a certain range. The alarm may provide either an audio and/or visual indication to the user that the resistance/impedance value has exceeded the outer predefined limits. Additionally, the unit may contain a ground fault indicator, and/or a tissue temperature monitor. The front panel 24 of the console 14 typically contains meters and displays that provide an indication of the power, frequency, etc., of the power delivered to the probe.

The console 14 may deliver a radiofrequency (RF) power output in a frequency range of 100 KHz-5 MHz. In the preferred embodiment, power is provided to the probe at a frequency in the range of 350 KHz. The time duration of each application of power to a particular location of tissue can be up to several seconds.

If the system incorporates temperature sensors, the console 14 may control the power such that the target tissue temperature is maintained to no more than approximately 100° C., to avoid necrosis of the tissue. The temperature sensors can be carried by the probe 12, incorporated into the electrodes 28 and 30, or attached within proximity to the electrodes 28 and 30.

If the system includes an impedance monitor, the power could be adjusted so that the target tissue impedance, assuming a probe 12 with a tip of length 460 um and diameter of 90 um, decreases by approximately 50% from an initial value that is expected to range between 1100 to 1800 ohm. If two or more electrodes are energized in parallel, the initial impedance values may be less than 1000 ohm. For bipolar applications, the initial impedance values may be higher, over 2000 ohms, under nominal circumstances. The console 14 could regulate the power down if, after an initial descent, the impedance begins to increase. Controls can be incorporated to terminate RF delivery if the impedance increases by a significant percentage from the baseline. Alternatively, or additionally, the console 14 could modulate the duration of RF delivery such that delivery is terminated only when the impedance exceeds a preset percentage or amount from a baseline value, unless an upper time limit is exceeded. Other time-modulation techniques, such as monitoring the derivative of the impedance, could be employed. Time-modulation could be based on physiologic parameters other than tissue impedance (e.g tissue water content, chemical composition, etc.)

FIG. 3 shows a typical voltage waveform that is delivered by the probe 12 to the skin. Each pulse of energy delivered by the probe 12 may be a highly damped sinusoidal waveform, typically having a crest factor (peak voltage/RMS voltage) greater than 5:1. Each highly damped sinusoidal waveform is repeated at a repetitive rate. The repetitive rate may range between 4-12 KHz and is preferably set at 7.5 KHz. Although a damped waveform is shown and described, other waveforms, such as continuous sinusoidal, amplitude, frequency or phase-modulated sinusoidal, etc. can be employed.

FIG. 4, shows the electrodes 28 and 30 inserted into a cornea. The pointed tips 32 and 34 of the electrodes 28 and 30, respectively, assist in the penetration of the cornea. The tips 32 and 34 are typically inserted until the bottom surface 38 of the housing 36 engages the cornea. The bottom surface 38 thus functions as a stop that limits the penetration depth of the electrodes 28 and 30. Although a stop is shown and described, it is to be understood that the probe 12 does not need to have a stop. The dielectric material of the stop minimizes the flow of current on the top layer of cornea. Minimizes current flow on the top layer improves the energy delivery efficiency of the system and reduces heat within the epithelium of the cornea.

The electrodes 28 and 30 should have a length that insures sufficient penetration into the stroma layer of the cornea. By way of example, the electrodes 28 and 30 may each have a length between 300 to 800 microns. The diameter of the each electrode 28 and 30 should be sufficient to provide the desired amount of energy but be small enough to not leave unsightly incision wounds. In one embodiment, the diameter of each electrode 28 and 30 is 90 microns. The electrodes 28 and 30 could carry, have embedded in it, or otherwise attached to it, specialized sensors (not shown), such as temperature sensors (e.g. thermocouples, thermistors, etc.), pressure sensors, etc. Although specific lengths and diameters have been disclosed, it is to be understood that the tip may have different lengths and diameters.

In operation, the a surgeon inserts the electrodes 28 and 30 into the cornea down into the stroma layer. The surgeon then activates the power unit to deliver energy to the first electrode 28. The energy flows from the first electrode 28, through the cornea and to the second electrode 30. The current generates heat that denatures the collagen tissue of the stroma.

Because the electrodes 28 and 30 are inserted into the stroma, it has been found that a power no greater than 1.2 watts for a time duration no greater than 1.0 seconds will adequately denature the corneal tissue to provide optical correction of the eye. However, other power and time limits, in the range of several watts and seconds, respectively, can be used to effectively denature the corneal tissue. Inserting the electrodes 28 and 30 into the cornea provides improved repeatability over probes placed into contact with the surface of the cornea, by reducing the variances in the electrical characteristics of the epithelium and the outer surface of the cornea.

FIG. 5 shows a pattern of denatured areas 50 that have been found to correct hyperopic or presbyopic conditions. A circle of 8, 16, or 24 denatured areas 50 are created about the center of the cornea, outside the visual axis portion 52 of the eye. The visual axis has a nominal diameter of approximately 5 millimeters. It has been found that 16 denatured areas provide the most corneal shrinkage and less post-op astigmatism effects from the procedure. The circles of denatured areas typically have a diameter between 6-8 mm, with a preferred diameter of approximately 7 mm. If the first circle does not correct the eye deficiency, the same pattern may be repeated, or another pattern of 8 denatured areas may be created within a circle having a diameter of approximately 6.0-6.5 mm either in line or overlapping.

The assignee of the present application provides instructional services to educate those performing such procedures under the service marks CONDUCTIVE KERATOPLASTY and CK. The bi-polar electrode assembly can be used to create two denatured spots in one application of energy. Simultaneous creation of denatured spots reduces the time required to perform the overall procedure. Additionally, the fixed distance between the electrodes 28 and 30 insures a uniform spacing between denatured spots.

The exact diameter of the pattern may vary from patient to patient, it being understood that the denatured spots should preferably be formed in the non-visionary portion 52 of the eye. Although a circular pattern is shown, it is to be understood that the denatured areas may be located in any location and in any pattern. In addition to correcting for hyperopia, the present invention may be used to correct astigmatic conditions. For correcting astigmatic conditions, the denatured areas are typically created at the end of the astigmatic flat axis. The present invention may also be used to correct procedures that have overcorrected for a myopic condition.

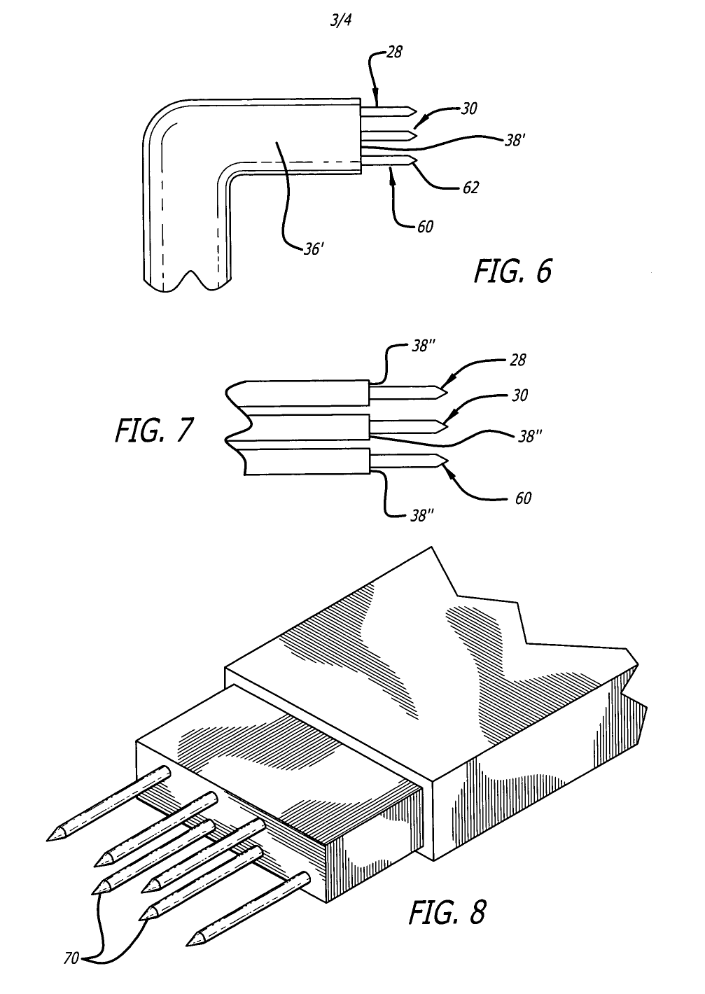

FIG. 6 shows an alternate embodiment of an electrode assembly that has a third electrode 60. The third electrode 60 may have a pointed tip 62 that extends from the housing 36′. The electrodes 28, 30 and 60 extend from a bottom surface 38′ of the housing 36′. The tri-polar tip can be used to simultaneously create three denatured spots with a single application of energy. In this embodiment energy can flow from both the first 28 and third electrodes 60 to the second electrode 30. The third electrode 60 may be separated from the second electrode 30 approximately 0.2 to 2.0 mm. Conversely, the system can be configured so that energy flows from the second electrode to the first and third electrodes, or any other combination of electrode current flow.

FIG. 7 shows another embodiment of an electrode assembly with separate stops 38″. Although a tri-polar assembly is shown, it is to be understood that a bi-polar assembly may have separate stops.

FIG. 8 shows another embodiment of a probe with a plurality of electrodes 70. The tips 70 may be connected to the console so that there are a number of bi-polar tip pairs. This embodiment allows for the simultaneous creation of multiple pairs of denatured spots.

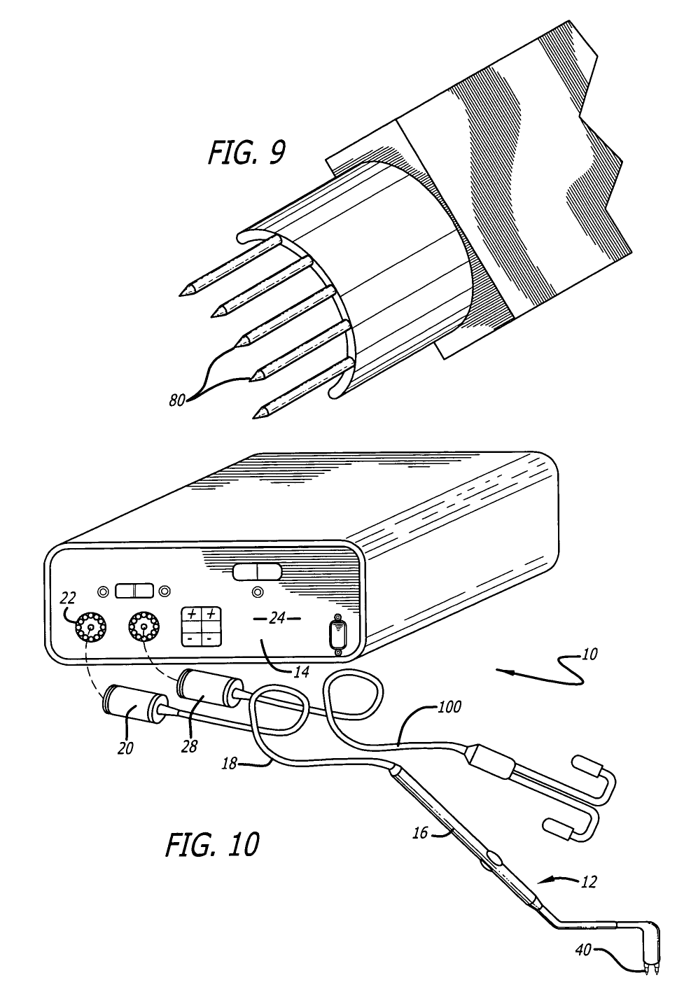

FIG. 9 shows another embodiment of a probe with a plurality of electrode tips 80 arranged in a radial pattern. This probe may also allow for the simultaneous creation of multiple denatured areas to reduce the time required to perform a procedure. The radial pattern may be a complete circle, a segment of a circle, or any other pattern.

FIG. 10 shows an alternate embodiment of a system with a ground element 100. The ground element 100 may be a lid speculum that is placed on the patients eye. In this embodiment energy flows from the electrodes to the ground element to denature corneal tissue.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

For example, although the delivery of radio frequency energy is described, it is to be understood that other types of non-thermal energy such as direct current (DC) and microwave can be transferred into the skin tissue through the probe.

By way of example, the console can be modified to supply energy in the microwave frequency range or the ultrasonic frequency range. By way of example, the probe may have a helical microwave antenna with a diameter suitable for delivery into the tissue. The delivery of microwave energy could be achieved with or without tissue penetration, depending on the design of the antenna. The system may modulate the microwave energy in response to changes in the characteristic impedance.

What is claimed is:

1. An intra-stroma probe that is used to denature corneal tissue, comprising:
a first electrode with a tip; and,
a second electrode that has a tip and is separated from said first electrode.

2. The probe of claim 1, wherein said first electrode extends from a first stop.

3. The probe of claim 1, wherein said second electrode extends from a second stop.

4. The probe of claim 1, further comprising a third electrode that has a tip, and is spaced from said first and second electrodes.

5. The probe of claim 4, wherein said third electrode extends from a third stop.

6. The probe of claim 1, further comprising a housing connected to said first and second electrodes, said housing provides a stop to limit a penetration depth of said first and second electrodes.

7. The probe of claim 2, wherein said first electrode extends from said first stop 300 to 800 microns.

8. The probe of claim 1, wherein said first and second electrodes are separated approximately 0.2 to 2.0 millimeters.

9. The probe of claim 4, wherein said second and third electrodes are separated approximately 0.2 to 2.0 millimeters.

10. An system that is used to denature corneal tissue, comprising:
a first electrode with a tip;
a second electrode that has a tip and is separated from said first electrode; and,
a power unit that delivers energy to said first electrode sufficient to denature tissue of a cornea.

11. The system of claim 10, wherein said first electrode extends from a first stop.

12. The system of claim 10, wherein said second electrode extends from a second stop.

13. The system of claim 10, further comprising a third electrode that has a tip, and is spaced from said first and second electrodes.

14. The system of claim 13, wherein said third electrode extends from a third stop.

15. The system of claim 10, further comprising a housing connected to said first and second electrodes, said housing provides a stop to limit a penetration depth of said first and second electrodes.

16. The probe of claim 11, wherein said first electrode extends from said first stop 300 to 800 microns.

17. The probe of claim 10, wherein said first and second electrodes are separated approximately 0.2 to 2.0 millimeters.

18. The probe of claim 13, wherein said second and third electrodes are separated approximately 0.2 to 2.0 millimeters.

19. The probe of claim 10, wherein said power unit delivers radio frequency energy to said first electrode.

20. The probe of claim 10, further comprising a hand piece that is coupled to said first and second electrodes and said power unit.

21. An system that is used to denature corneal tissue, comprising:
electrode means for insertion into the cornea and delivery of energy to the cornea to denature corneal tissue; and,
a power unit that provides a sufficient amount of energy to said electrode means to denature the corneal tissue.

22. The system of claim 21, wherein electrode means includes a first electrode with a tip.

23. The system of claim 22, wherein said electrode means includes a second electrode with a tip.

24. The system of claim 23, wherein said electrode means includes a third electrode with a tip.

25. The system of claim 24, wherein said first, second and third electrodes each extend from a stop.

26. The system of claim 23, further comprising a housing connected to said first and second electrodes, said housing provides a stop to limit a penetration depth of said first and second electrodes.

27. The system of claim 24, wherein said first, second and third electrodes extend from said stop 300 to 800 microns.

28. The system of claim 23, wherein said first and second electrodes are separated approximately 0.2 to 2.0 millimeters.

29. The system of claim 24, wherein said first, second and third electrodes are separated approximately 0.2 to 2.0 millimeters.

30. The system of claim 21, wherein said power unit delivers radio frequency energy to said electrode means.

31. The system of claim 21, further comprising a hand piece that is coupled to said electrode means and said power unit.

32. The system of claim 21, wherein said electrode means includes a first electrode and a second electrode that is co-planar with and spaced from first electrode.

33. An intra-stroma probe that is used to denature corneal tissue, comprising:
a first electrode; and,
a second electrode that is essentially co-planar with and separated from said first electrode.

34. The probe of claim 33, wherein said first electrode includes a tip and extends from a first stop.

35. The probe of claim 33, wherein said second electrode includes a tip and extends from a second stop.

36. The probe of claim 33, further comprising a third electrode that is co-planar with and spaced from said first and second electrodes.

37. The probe of claim 36, wherein said third electrode includes a tip and extends from a third stop.

38. The probe of claim 33, further comprising a housing connected to said first and second electrodes, said housing provides a stop to limit a penetration depth of said first and second electrodes.

39. The probe of claim 34, wherein said first electrode extends from said first stop 300 to 800 microns.

40. The probe of claim 33, wherein said first and second electrodes are separated by approximately 0.2 to 2.0 millimeters.

41. The probe of claim 36, wherein said second and third electrodes are separated by approximately 0.2 to 2.0 millimeters.

42. An system that is used to denature corneal tissue, comprising:
a first electrode;
a second electrode that is essentially co-planar with and separated from said first electrode; and,
a power unit that delivers energy to said first electrode sufficient to denature tissue of a cornea.

43. The system of claim 42, wherein said first electrode includes a tip and extends from a first stop.

44. The system of claim 42, wherein said second electrode includes a second tip and extends from a second stop.

45. The system of claim 42, further comprising a third electrode that is co-planar with and spaced from said first and second electrodes.

46. The system of claim 45, wherein said third electrode includes a tip and extends from a third stop.

47. The system of claim 42, further comprising a housing connected to said first and second electrodes, said housing provides a stop to limit a penetration depth of said first and second electrodes.

48. The system of claim 43, wherein said first electrode extends from said first stop 300 to 800 microns.

49. The system of claim 42, wherein said first and second electrodes are separated by approximately 0.2 to 2.0 millimeters.

50. The system of claim 45, wherein said first, second and third electrodes are separated from each other by approximately 0.2 to 2.0 millimeters.

51. The system of claim 42, wherein said power unit delivers radio frequency energy to said first electrode.

52. The system of claim 42, further comprising a hand piece that is coupled to said first and second electrodes and said power unit.

53. A method for denaturing a cornea, comprising:
inserting a first electrode and a second electrode into a cornea; and,
delivering energy that flows from the first electrode, through the cornea and into the second electrode.

54. The method of claim 53, wherein the first and second electrodes are inserted until a stop engages the cornea.

55. The method of claim 53, further comprising inserting a third electrode with the first and second electrodes and delivering energy that flows between the first, second and third electrodes.

56. The method of claim 53, wherein the first and second electrodes are inserted in an area of the cornea that is 6 to 8 millimeters about a center of the cornea.

57. The method of claim 53, wherein the first and second electrodes are inserted into the cornea in a circular pattern.

58. A method for denaturing a cornea of a patient, comprising:
grounding a patient with a ground element;
inserting a first electrode and a second electrode into a cornea; and,
delivering energy that flows from the first and second electrodes, through the cornea and into the ground element.

59. The method of claim 58, wherein the first and second electrodes are inserted until a stop engages the cornea.

60. The method of claim 58, further comprising inserting a third electrode into the cornea and delivering energy that flows from the third electrode, through the cornea and into the ground element.

61. The method of claim 58, wherein the first and second electrodes are inserted in an area of the cornea that is 6 to 8 millimeters about a center of the cornea.

62. The method of claim 58, wherein the first and second electrodes are inserted into the cornea in a circular pattern.
